(19) United States
(12) Patent Application Publication
(10) Pub. No.: US 2025/0369577 A1
(43) Pub. Date: Dec. 4, 2025

(54) RECESSED LIGHTING WITH UPPER VISUAL HEMISPHERE CEILING WASH

(71) Applicant: KORRUS, INC., Los Angeles, CA (US)

(72) Inventor: Paul Kenneth Pickard, Los Angeles, CA (US)

(21) Appl. No.: 19/244,768

(22) Filed: Jun. 20, 2025

Related U.S. Application Data
(63) Continuation of application No. PCT/US23/85372, filed on Dec. 21, 2023.
(60) Provisional application No. 63/434,322, filed on Dec. 21, 2022.

(51) Int. Cl. F21S 8/02; F21V 23/00; F21Y 113/13; F21Y 115/10

(52) U.S. Cl. CPC F21S 8/026; F21V 23/003; F21Y 2113/13; F21Y 2115/10

(57) ABSTRACT

A lighting system comprising: (a) a housing having an axis and configured for mounting in a ceiling; (b) a first light source in said housing configured for emitting first light axially relative to said housing; and (c) a second light source in said housing configured for emitting second light radially relative to said housing such that, when said housing is mounted in said ceiling, an area of said ceiling around the perimeter of said housing is illuminated.

### CROSS REFERENCE TO RELATED APPLICATIONS

The present patent application is a continuation of International Application No. PCT/US23/85372, filed Dec. 21, 2023, which claims the benefit of U.S. Provisional Patent Application 63/434,322, filed Dec. 21, 2022, the entire disclosures of each of which are hereby incorporated by reference.

### FIELD OF INVENTION

The present invention is directed generally to enhancing indoor lighting, and, more specifically, to adding overhead light to recessed lighting to optimize beneficial biological and psychological impacts on a user.

### BACKGROUND

In a previous disclosure (US Patent Application Publication 20210215317A1), a system was described for providing an uplight/downlight suspended linear fixture that provided the appropriate CCT and spectral content for the time of day in the down direction and provided the ability to create a simulated sky color in the up direction. This allowed for indirect lighting with decreased blue in the late afternoon evening (when the simulated sky would be trending orange/red) and increased blue in the morning and early afternoon (when the simulated sky would look sky blue.) This is an effective solution for providing improved differentials in day/night EML as well as color cues for occupants in commercial settings with white ceilings. However, the late afternoon/evening impact of such a system is limited as most employees do not work late into the evening.

A similarly functioning system could have substantial impacts on occupant circadian health in a home environment (due to much greater late afternoon and nighttime occupancy), but the suspended linear fixture is not a preferred method for lighting apartments, houses, and home offices. The ubiquitous lighting fixture for these spaces is the recessed downlight.

As its name implies, the recessed downlight is a fixture which traditionally is recessed above the ceiling plane, providing a cone (with a reflective trim) or bloom (with a matte white trim) of light. At a close enough distance from the fixture, depending on ceiling height and light source recess depth, the light source is visible to the occupant. Recessed downlights in the middle of rooms tend to illuminate furniture or walkways in the home environment. Recessed downlights near the wall tend to illuminate the wall with sharply defined cones of light (reflective trims) or soft washes of light (matte white trims.) There are even optical treatments of downlights that are designed specifically to create very even vertical illumination called “wall washers.”

Recessed downlights are well suited for illuminating horizontal surfaces (floor and furniture) and walls—but typically great care is taken to not have such fixture appear bright from above. More specifically, the bright light source in the downlight contrasts with the relatively dark ceiling, and thus tends to increase the perception of glare.

However, Applicant recognizes that for optimal circadian lighting, early morning to afternoon lighting should emphasis light in the upper visual hemisphere (eyeline and above), while in the evening the upper visual hemisphere should remain as dark as possible, or be lit at very low levels with low blue light. In other words, the human brain is influenced disproportionately by light above the eyeline than below, so providing stimulating light in the morning above the eyeline and soothing low CS lighting below the eyeline in the evening is more beneficial than varying the downlights with high and low CS light.

While downlights placed away from walls can provide a reasonable evening illumination scenario, especially with “warm dim” functionality that allows for warmer CCTs at lower light levels, there are no downlights designed specifically to generate good light in the upper visual hemisphere, and certainly none that could transition from excellent, high blue upper hemisphere lighting during the early morning to afternoon, then transition to lower hemisphere low blue lighting in the evening. Accordingly, Applicant recognizes a need for such lighting. The present invention fulfills this need, among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

Broadly, the present invention provides, in one embodiment, a recessed downlight designed specifically to generate light in the upper visual hemisphere, which transitions from high blue lighting during the early morning to afternoon, and then transition to low blue lighting in the evening. More specifically, the recessed downlight is configured with two light sources, one source emitting light downward, which varies in EML and/or CCT (or color) throughout the day, and a second source emitting light radially to wash the ceiling in light, which varies in EML and/or CCT (or color) to provide a circadian-friendly upper visual hemisphere.

In one embodiment, the lighting system comprises a housing having an axis and a housing edge, and is configured for mounting in a ceiling. In the housing is a first light source configured for emitting first light axially relative to said housing, and a second light source configured for emitting second light radially relative to said housing such that, when said housing is mounted in said ceiling, an area of said ceiling around the housing edge of said housing is illuminated by the second light.

### BRIEF DESCRIPTION OF FIGURES

FIG. 1 shows a schematic cross-section of one embodiment of the lighting system of the present invention.

FIG. 2 shows a schematic bottom view of the lighting system of FIG. 1 eliminating an area on a ceiling plane.

FIG. 3 shows a schematic of an alternative embodiment of a lighting system of the present invention.

### DETAILED DESCRIPTION

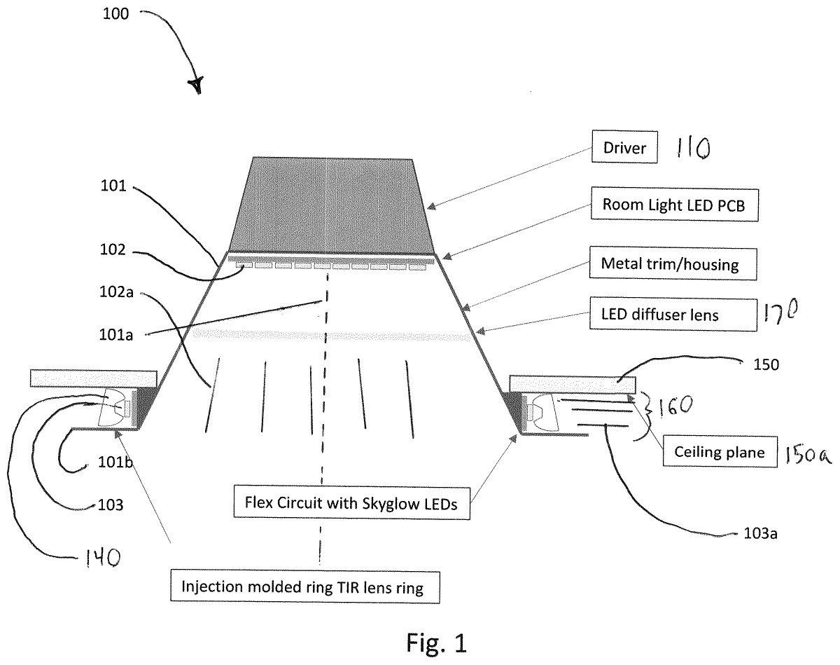

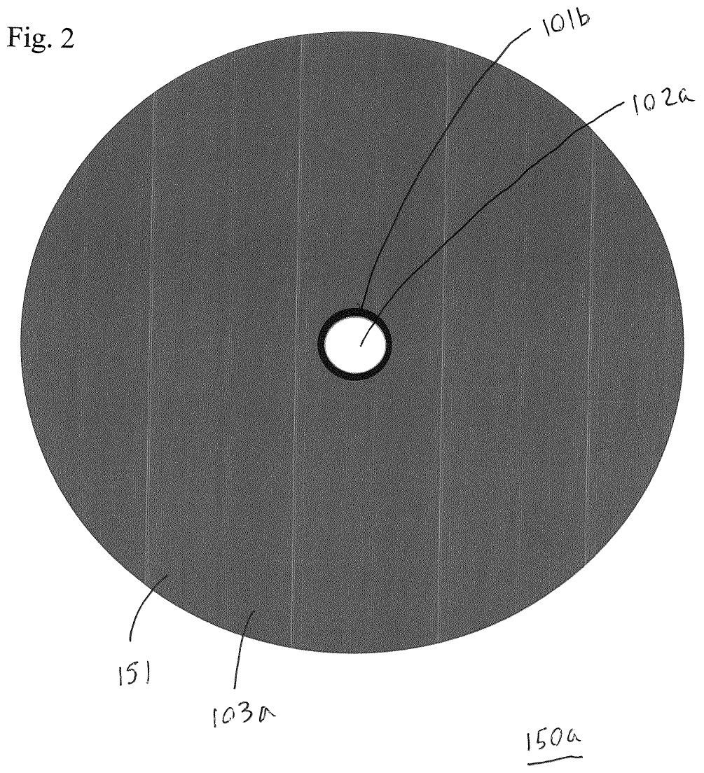

Referring to FIGS. 1 and 2, one embodiment of a lighting system 100 of the present invention is shown. Lighting system 100 comprises a housing 101 having an axis 101a and a housing edge 101b, and is configured for mounting in a ceiling 150. In the housing 101 is a first light source 102 configured for emitting first light 160 axially relative to said housing, and a second light source 103 configured for emitting second light 161 radially relative to said housing such that, when said housing is mounted in said ceiling, an area 151 of said ceiling around the housing edge 101b of said housing is illuminated by the second light 161. These elements are considered in greater detail below, and with respect to alternative selected embodiments.

### Control

In one embodiment, the lighting system 100 further comprises driver circuitry 110 for driving and controlling at least one of said first light source 102 or said second light source 103. In one embodiment, the drive circuitry is configured to control said first and second light sources independently as described below. Such drive circuitry is well-known in the art and will not be considered in detail herein.

In one embodiment, the controller is configured to create custom scenes triggered by astronomical or individual Circadian clocks. In one embodiment, the control circuitry is configured to control said second light source to emit said second light to illuminate said area 151 corresponding to at least one of (a) said first light, (b) the circadian rhythm of a user, or (c) time of day in location of said system.

For example, in one embodiment, the first light source provides high EML cool white light early in the day, high color quality 2700K to 3500K white in the afternoons/early evenings, and could reduce to, for example, 2400K in the evenings—all dimmable via an ELV dimmer to custom selectable warmer CCTs which would vary throughout the day. Coordinating with the first light source, the second light source, in one embodiment, can either mirror the first light source CCT behavior (providing an indirect source of the same CCT/EML), or, in another embodiment, create a “skyglow” scene—starting at dim red/orange in the early morning, progressing through pale yellow to pale blue throughout the day, transitioning to back to red/orange in the evening, and eventually turning off in the later evening (to provide overhead darkness to help facilitate Dim Light Melatonin Onset.) In one embodiment, either configuration is influenced by the astronomical clock or individual Circadian clock, which may be synced to the output of the first light source.

### First Light

The first light source functions to provide downlight to illuminate horizontal surfaces (floor and furniture) and walls. In one embodiment, said control circuitry is configured to control said first light source to emit said first light with varying EML. In one embodiment, said first light has at least a high-EML second light and a low-EML second light. In one embodiment, said high-EML first light has a high-EML spectrum power distribution (SPD) having a high-EML overall power from 350 nm-800 nm, and a high-EML blue power from 440 nm-510 nm, wherein said high-EML blue power is at least 20% of said high-EML overall power, and wherein said low-EML first light has a low-EML spectrum power distribution (SPD) having a low-EML overall power from 350 nm-800 nm, and a low-EML blue power from 440 nm-510 nm, wherein said low-EML blue power is no greater than 2% of said low-EML overall power.

In one embodiment, said control circuitry is configured to transition between said high-EML first light and said a low-EML first light by incremental changes in CCT. In one embodiment, said incremental changes in CCT are no greater than 250K/min, or 200K/min, or 150K/min, or 100K/min. In one embodiment, said high-EML first light has a CCT of 4000 to 6000K, and said a low-EML first light has a CCT of 1500 to 2700K. In one embodiment, said control circuitry is configured to control said first light source such that said first light transitions between said high-EML first light and said low-EML first light within a 24 hour period. In one embodiment, the light source is scheduled by a smartphone application that would provide clock time to the fixture, allowing for gradual adjustments of non-dimmed CCT (and target CCT for “dim to warm”) throughout the day.

Various configurations of one or more light sources may be used to achieve the different EMLs and CCTs of the second light described above. In one embodiment, standard low-glare optics are used for providing traditional downlight distributions combined with a custom light source. In one embodiment, one light source can provide high-EML, cool CCT (5700K) white light and “warm dimming” to 3000K, high color quality below-black-body color point during the early morning through afternoon. In one embodiment, another light source can provide a 3000K, high color quality, below-black-body color point during the late afternoon and evening, “warm dimming” to 1800K. In one embodiment, the 1800K light source and 5700K light source can be disposed on the same COB. In one embodiment, the 1800K light source can be constrained to the center of the COB, allowing for narrower beam angles of lighting distribution as the CCT gets warmer, thereby providing for less prominent wall lighting during the late afternoon and evening. In one embodiment, the first light source is a single light source, such as, for example, Ecosense Vigor. Still other embodiments of the light source will be known to those of skill in the art in light of this disclosure.

### Second Light

The second light source functions to provide upper hemisphere lighting (i.e. light above the eyeline). In one embodiment, the second light source is configured to emit second light emulating sky color throughout the day according to an astronomical clock or individual circadian clock. In one embodiment, the second light progresses from pale yellow to pale blue to red/orange and eventually turning off within a 24 hour period. In one embodiment, the second light is red-orange of sunrise/sunset or pale blue of a clear blue sky, and, in one embodiment, also has intervening colors (e.g. yellows and greens).

In one embodiment, the second light source is controlled independently from the first light source, although synchronized to provide Circadian-appropriate lighting throughout the day.

In one embodiment, the second light source comprises an optical construct 140 to moderate the emitted second light. For example, in one embodiment, the optical construct 140 may achieve effective color mixing to provide a uniform color of the second light output from the second light source. In another embodiment, the optical construct may be configured to provide uniform ceiling illumination in proximity to the housing edge.

In one embodiment, the second light source is configured to emit second light in an area 151 on the ceiling plane 150a around the housing edge 101b of the housing 101 as shown in FIG. 2. In one embodiment, said area has a consistent illumination portion wherein the ratio of average lux to min lux in said consistent illumination portion is no greater than 3, wherein said consistent illumination portion has a diameter of at least 2 ft. In one embodiment, said diameter is at least 3 ft.

In one embodiment, the lighting system is configured to emit second light to emulate a sky color having a high-EML in the morning, and a low-EML second light in the evening. In one embodiment, said high-EML second light has a relatively high light intensity, and said low-EML second light has a relatively low light intensity. In one embodiment, said second light is synchronized to said first light such that said second light has an EML that varies with the EML of the first light. In one embodiment, said second light has a low EML second light which is lower than the low EML first light. In one embodiment, when the first light source switches to a low EML light, the second light source is switched off. Those of skill in the art in light of this disclosure will appreciate that the various modes of the first and second light sources can be mixed and matched based on a user's preferences.

### Physical Configuration

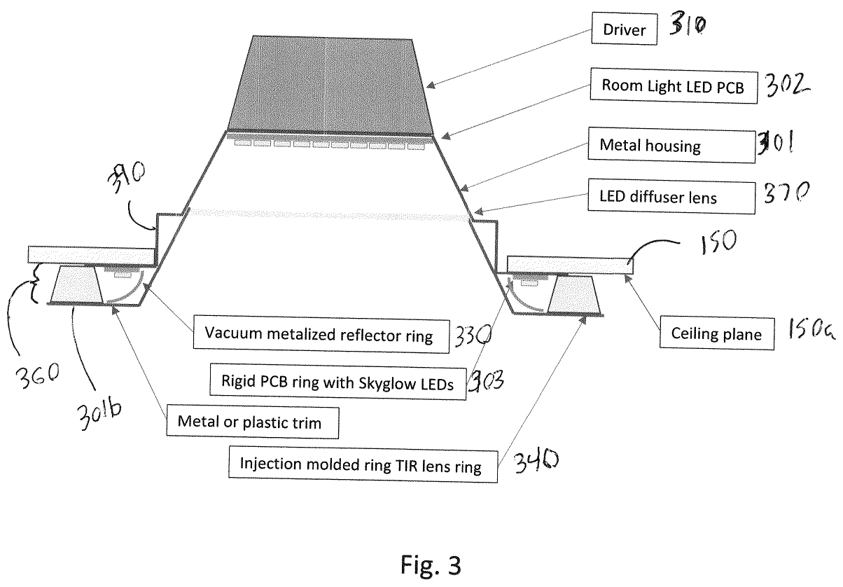

Various physical configurations of the lighting system of the present invention are possible. For example, two different embodiments are shown in FIGS. 1 and 3. In these examples, the downlight housings 101, 301 are similar, and extend beyond the ceiling plane 150a to provide a small gap 160, 360 between the housing edge 101b, 301b and ceiling plane 150a. It is generally preferred to minimize the size of the gap, thereby minimizing the extent to which the housing extends from the ceiling plane. In one embodiment, said gap is no more than 20 mm, or no more than 15 mm, or no more than 10 mm from said ceiling when installed.

Embodiments of FIGS. 1 and 3 also share other common features, including, for example, the upper portion of the housing containing the driver circuitry 110, and the first light source 102, 302, which, in these embodiments, comprises LEDs mounted on a PCB. Both embodiments also include a LED diffuser lens 170, 370 to diffuse light from the first light source to form said first light 102a.

The embodiments of FIGS. 1 and 3 differ in the configuration of the second light source. Specifically, in the embodiment of FIG. 1, the second light source 103 comprises a series of LEDs mounted vertically within the gap 160, while, in embodiment of FIG. 3, the second light source 303 comprises a series of LEDs mounted horizontally in the gap 360 on a mounting flange 390.

In both these embodiments, injection molded TIR rings 140, 340 are disposed in the gap in front of the second light source and are designed to create an asymmetric distribution with peak output ˜2 degrees above horizontal provides the remainder of the beam shaping necessary to achieve a low contrast illuminated spot on the ceiling. The angle from vertical for the exit surface of the TIR ring is designed to insure that minimal light directly from the LED escapes at an angle lower than horizontal. It should be noted that TIR ring 140 and TIR ring 340 are different in shape due to the different configuration of the second light source in each embodiment. Such optics are obvious to those of skill the art will not be described here in detail.

Referring to FIG. 3, in this embodiment, a metalized reflector ring 330 is disposed in the gap 360 between the housing edge 101b and the ceiling plane 150a. This configuration provides reflection from the LEDs in an angle of incidence range where a TIR would not be effective. With plasma-treated silver, the reflectance for this ring could be 90%+. Any heat associated with absorbed light would be conducted to a room side heat radiator in the housing itself.

While this is one embodiment of such an optical system, one skilled in the art would recognize there are other methods (light guides, purely reflective optics, and others) that could achieve the same outcome.

Thus, FIGS. 1 and 3 show a lighting fixture suitable for use in residential and multi-family construction—capable of providing not only the time appropriate spectrum from the traditional downlight element, but also the spatially correct visual cues for alertness during the day (pale blue in the upper visual hemisphere) and for the preservation of Melatonin production at night (darkness in the upper visual hemisphere.)

These and other advantages may be realized in accordance with the specific embodiments described as well as other variations. It is to be understood that the above description is intended to be illustrative, and not restrictive. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled.

What is claimed is:

1. A lighting system comprising:
a housing having an axis and configured for mounting in a ceiling;
a first light source in said housing configured for emitting first light axially relative to said housing; and
a second light source in said housing configured for emitting second light radially relative to said housing such that, when said housing is mounted in said ceiling, an area of said ceiling around the perimeter of said housing is illuminated.

2. The lighting system of claim 1, wherein said control circuitry independently controls said first light source and said second light source.

3. The lighting system of claim 2, wherein said control circuitry is configured to control said first light source to emit said first light with varying EML.

4. The lighting system of claim 3, wherein said first light has at least a high-EML second light and a low-EML second light.

5. The lighting system of claim 3, wherein said high-EML first light has a high-EML spectrum power distribution (SPD) having a high-EML overall power from 350 nm-800nm, and a high-EML blue power from 440 nm-510 nm, wherein said high-EML blue power is at least 20% of said high-EML overall power, and wherein said low-EML first light has a low-EML spectrum power distribution (SPD) having a low-EML overall power from 350 nm-800 nm, and a low-EML blue power from 440 nm-510 nm, wherein said low-EML blue power is no greater than 2% of said low-EML overall power.

6. The lighting system of claim 5, wherein said control circuitry is configured to transition between said high-EML first light and said a low-EML first light by incremental changes in CCT.

7. The lighting system of claim 6, wherein said incremental changes in CCT are no greater than 100K/min.

8. The lighting system of claim 4, wherein said high-EML first light has a CCT of 4000 to 6000K, and said a low-EML first light has a CCT of 1500 to 2700K.

9. The lighting system of claim 4, wherein said control circuitry is configured to control said first light source such that said first light transitions between said high-EML first light and said low-EML first light within a 24 hour period.

10. The lighting system of claim 2, wherein said control circuitry is configured to control said second light source to emit said second light to illuminate said area corresponding to at least one of (a) said first light, (b) the circadian rhythm of a user, or (c) time of day in location of said system.

11. The lighting system of claim 1, wherein said second light illuminates said area in a sky color

12. The lighting system of claim 11, wherein said second light progresses from pale yellow to pale blue to red/orange and eventually turning off within a 24 hour period.

13. The lighting system of claim 12, wherein said sky color has a high-EML second light in the morning, and a low-EML second light in the evening.

14. The lighting system of claim 13, wherein said high-EML second light has a relatively high light intensity, and said low-EML second light has a relatively low light intensity.

15. The lighting system of claim 11, wherein said second light is synchronized to said first light such that said second light has an EML that varies with the EML of the first light.

16. The lighting system of claim 15, wherein said second light has a low EML second light which is lower than the low EML first light.

17. The lighting system of claim 16, wherein said second light is off when said first light is in a low EML mode.

18. The lighting system of claim 1, wherein said area has a consistent illumination portion wherein the ratio of average lux to min lux in said consistent illumination portion is no greater than 3, wherein said consistent illumination portion has a diameter of at least 2 ft.

19. The lighting system of claim 1, wherein said housing protrudes no more than 15 mm from said ceiling when installed.

20. The lighting system of claim 1, further comprising at least one optical element optically coupled to said second light source such that said second light is diffused light.
